(19) United States
(12) Patent Application Publication
(10) Pub. No.: US 2007/0042429 A1
(43) Pub. Date: Feb. 22, 2007

(54) Assay for differentiating Alzheimer's and Alzheimer's-like disorders

(76) Inventors: Ira Leonard Goldknopf, The Woodlands, TX (US); Essam Ahmed Sheta, The Woodlands, TX (US); Brian R. Folsom, Chanhassen, MN (US); Stanley H. Appel, Houston, TX (US); Albert A. Yen, Pearland, TX (US)

Correspondence Address: Power3 Medical Products, Inc., 3400 Research Forest Drive, The Woodlands, TX, 77381, US

(21) Appl. No.: 11/503,881

(22) Filed: Aug. 14, 2006

Related U.S. Application Data
(60) Provisional application No. 60/708,992, filed on Aug. 17, 2005.

(51) Int. Cl. G01N 33/53

(52) U.S. Cl. 435/7.1; 204/450

(57) ABSTRACT

The invention relates to an assay for discriminating between Alzheimer's disease (AD) patients, patients with AD-Like disorders that express symptoms like AD, and non-demented age-matched controls (Normal). The method is based on the use of 2-dimensional (2D) gel electrophoresis to separate the complex mixture of proteins found in blood serum, the quantitation of a group of identified biomarkers, and the biostatistical analysis of the concentration of the identified biomarkers to differentiate patients having AD from Normals and patients having other AD-Like disorders.

### CROSS REFERENCE TO RELATED APPLICATIONS

This application claims priority to U.S. Provisional patent application Ser. No. 60/708,992 filed on Aug. 17, 2005 and entitled “Assay for differentiating Alzheimer's and Alzheimer's-Like Disorders” by inventors Ira L. Goldknopf, et al.

### BACKGROUND OF THE INVENTION

1. Field of the Invention

The invention relates to a method for discriminating between patients having Alzheimer's disease (AD), patients with AD-Like disorders that express symptoms Like AD, and normal age-matched individuals. The method is based on the use of 2-dimensional (2D) gel electrophoresis to separate the complex mixture of proteins found in blood serum and the quantitation of a group of identified biomarkers to differentiate patients having AD from patients having other AD-Like disorders and from normal individuals.

2. Description of the Related Art

Alzheimer's disease (AD) is a progressive brain disorder that gradually destroys a person's memory and ability to learn, reason, make judgments, communicate and carry out daily activities. As AD progresses, individuals may also experience changes in personality and behavior, such as anxiety, suspiciousness or agitation, as well as delusions or hallucinations. An estimated 4.5 million Americans have AD. The number of Americans with AD has more than doubled since 1980 and is predicted to reach from 11.3 million to 16 million patients (Hebert, L. E., et al. 2003. Archives of Neurology 60(8): 1119-1122.

Presently, the diagnosis of AD is a clinical one. There is no single test that can provide diagnostic certainty. The usual diagnostic process consists of a full medical history, a comprehensive physical and neurological examination, as well as assessing a patient's cognitive status. Cognitive impairment is typically tested using standardized cognitive screening test call the Mini Mental State Examination (MMSE). A patient's MMSE score is generally combined with clinical features and laboratory test results to classify the severity of AD as Normal, Mild, Moderate, or Severe.

There is a tremendous need for a definitive diagnostic test. Clinicians have long sought a diagnostic test to confirm the diagnosis of Alzheimer's disease (AD) and distinguish it from other AD-Like disorders that display similar symptoms. A definitive diagnostic test based on disease biomarkers, rather than a clinical and cognitive evaluation, may provide physicians with earlier treatment decisions that may improve patient outcomes.

Proteomics is a new field of medical research wherein proteins are identified and linked to biological functions, including roles in a variety of disease states. With the completion of the mapping of the human genome, the identification of unique gene products, or proteins, has increased exponentially. In addition, molecular diagnostic testing for the presence of certain proteins already known to be involved in certain biological functions has progressed from research applications alone to use in disease screening and diagnosis for clinicians. However, proteomic testing for diagnostic purposes remains in its infancy. There is, however, a great deal of interest in using proteomics for the elucidation of potential disease biomarkers.

Detection of abnormalities in the genome of an individual can reveal the risk or potential risk for individuals to develop a disease. The transition from risk to emergence of disease can be characterized as an expression of genomic abnormalities in the proteome. Thus, the appearance of abnormalities in the proteome signals the beginning of the process of cascading effects that can result in the deterioration of the health of the patient. Therefore, detection of proteomic abnormalities at an early stage is desired in order to allow for detection of disease either before it is established or in its earliest stages where treatment may be effective.

Recent progress using a novel form of mass spectrometry called surface enhanced laser desorption and ionization time of flight (SELDI-TOF) for the testing of ovarian cancer and Alzheimer's disease has led to an increased interest in proteomics as a diagnostic tool (Petrocoin, E. F. et al. 2002. Lancet 359:572-577; Lewczuk, P. et al. 2004. Biol. Psychiatry 55:524-530). Furthermore, proteomics has been applied to the study of breast cancer through use of 2D gel electrophoresis and image analysis to study the development and progression of breast carcinoma in patients and in plasma from Alzheimer's disease patients (Kuerer, H. M. et al. 2002. Cancer 95:2276-2282; Ueno, I. et al. 2000. Electrophoresis 21:1832-1845). In the case of breast cancer, breast ductal fluid specimens were used to identify distinct protein expression patterns in bilateral matched pair ductal fluid samples of women with unilateral invasive breast carcinoma.

Detection of biomarkers is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to raise protective antibodies or in a kit to identify the presence or absence of the biomarker in individuals who may have been exposed to mustard poisoning. U.S. Pat. No. 6,326,209 B1 discloses measurement of total urinary 17 ketosteroid-sulfates as biomarkers of biological age. U.S. Pat. No. 6,693,177 B1 discloses a process for preparation of a single biomarker specific for 0-acetylated sialic acid and useful for diagnosis and outcome monitoring in patients with lymphoblastic leukemia.

There is a tremendous need for a definitive diagnostic test to confirm the diagnosis of Alzheimer's disease (AD) and distinguish it from other AD-Like disorders that display similar symptoms but have different treatment options and prognosis. Clinicians have long sought such a diagnostic test in hopes of providing earlier treatment decisions and improved patient outcomes.

### SUMMARY OF THE INVENTION

The present invention is a diagnostic assay for differentiating patient's having Alzheimer's disease (AD) from patients with AD-Like disorders, and from non-demented normal controls. The method comprises collecting a biological sample from a patient having symptoms consistent with AD, quantitating up to 47 protein biomarkers identified as related to AD or AD-Like disorders, and determining whether or not the patient has AD or an AD-Like disorder based on the statistical analysis of the quantity of the selected protein biomarkers.

One aspect of the present invention is a method for screening a patient for AD or AD-Like disorders. The method includes: collecting a serum sample from a patient having symptoms consistent with AD, separating the proteins in the serum sample by 2D gel electrophoresis, quantitating a panel of protein biomarkers, and determining whether or not the patient has a AD or an AD-Like disorder based on the quantity of those biomarkers in the patient's serum.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the methods for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof reference is now made to the following descriptions taken in conjunction with the accompanying drawings, in which:

FIG. 1: a representative 2D gel electrophoretic image of human serum proteins with 47 biomarkers marked and numbered.

FIG. 2: a canonical plot of AD, AD-Like and Normal data. The data show multivariate means of all biomarkers and ratios in the two dimensions that best separate the three groups.

FIG. 3: Box and Whisker profile of key biomarkers used to discriminate among AD, AD-Like and Normal.

FIG. 4A: Box and Whisker profile of selected biomarker sum and ratios used to discriminate among AD, AD-Like and Normal.

FIG. 4B: Box and Whisker profile of selected biomarker ratios used to discriminate among AD, AD-Like and Normal.

Table 1: Mean±Standard Deviation (SD) of the 47 biomarkers in the sera of AD, AD-Like and Normal individuals. Values are expressed as PPM.

Table 2: Mean±Standard Deviation (SD) of the selected biomarker sum and ratios used in the discriminant function analysis.

Table 3: Discriminant Analysis of AD (n=40) and AD-Like (n=24) patients using 47 biomarkers.

Table 4: Discriminant Analysis of AD (n=40) AD-Like (n=24) patients compared to Normal (n=33) using 47 biomarkers.

### DESCRIPTION OF THE PREFERRED EMBODIMENTS

The present invention is a diagnostic test for differentiating individuals with Alzheimer's disease (AD) and patients with AD-Like disorders that express symptoms Like AD. The method is based on the use of 2-dimensional (2D) gel electrophoresis to separate the complex mixture of proteins found in blood serum and the quantitation of a group of identified biomarkers to differentiate patients having AD from patients having other AD-Like disorders.

In the context of the present invention an “AD-Like disorder” would include individuals with symptoms consistent with AD, including without limitation, memory loss, confusion, depression, erratic behavior, and/or belligerence. There are six main anatomical categories of AD-Like disorders including:

1. Stroke (e.g., Multi Infarct Dementia)

2. Depression

3. Infections

4. Drug Related Symptoms

5. Mild Cognitive Impairment

6. Parkinson's Disease (PD)

In the context of the present invention, the “protein expression profile” corresponds to the steady state level of the various proteins in biological samples that can be expressed quantitatively. These steady state levels are the result of the combination of all the factors that control protein concentration in a biological sample. These factors include but are not limited to: the rates of transcription of the genes encoding the hnRNAs; the rates of processing of the hnRNAs into mRNAs; the splicing variations during the processing of the hnRNAs into mRNAs which govern the relative amounts of the protein isoforms; the rates of processing of the various mRNAs by 3′-polyadenylation and 5′-capping; the rates of transport of the mRNAs to the sites of protein synthesis; the rate of translation of the mRNA's into the corresponding proteins; the rates of protein post-translational modifications, including but not limited to phosphorylation, nitrosylation, methylation, acetylation, glycosylation, poly-ADP-ribosylation, ubiquitinylation, and conjugation with ubiquitin Like proteins; the rates of protein turnover via the ubiquitin-proteosome system and via proteolytic processing of the parent protein into various active and inactive subcomponents; the rates of intracellular transport of the proteins among compartments such as but not limited to the nucleus, the lysosomes, golgi, the membrane, and the mitochondrion; the rates of secretion of the proteins into the interstitial space; the rates of secretion related protein processing; and the stability and rates of proteolytic processing and degradation of the proteins in the biological sample before and after the sample is taken from the patient.

In the context of the present invention, a “biomarker” corresponds to a protein or protein fragment present in a biological sample from a patient, wherein the quantity of the biomarker in the biological sample provides information about whether the patient exhibits an altered biological state such as AD or an AD-Like disorder.

A “control” or “normal” sample is a sample, preferably a serum sample, taken from an individual with no known disease, particularly without a neuromuscular disease.

The method of the present invention is based on the quantification of specified proteins. Preferably the proteins are separated and identified by 2D gel electrophoresis. 2D gel electrophoresis has been used in research laboratories for biomarker discovery since the 1970's (Orrick, L. R. et al. 1973. Proc. Natl. Sci. U.S.A. 70:13 16-1320; Goldknopf, I. L. et al. 1975. J. Biol Chem. 250:71282-7187; O'Farrell, P. et al. 1975. J. Biol. Chem. May 250:4007-4021; Anderson, L. and Anderson, N. G. 1977. Proc. Natl. Acad. Sci. U.S.A. 74:5421-5425; Goldknopf, I. L. and Busch, H. 1977. Proc. Natl. Acad. Sci. USA 74:864-868). In the past, this method has been considered highly specialized, labor intensive and non-reproducible.

Only recently with the advent of integrated supplies, robotics, and software combined with bioinformatics has progression of this proteomics technique in the direction of diagnostics become feasible. The promise and utility of 2D gel electrophoresis is based on its ability to detect changes in protein expression and to discriminate protein isoforms that arise due to variations in amino acid sequence and/or post-synthetic protein modifications such as phosphorylation, nitrosylation, ubiquitination, conjugation with ubiquitin-Like proteins, acetylation, and glycosylation. These are important variables in cell regulatory processes involved in disease states.

There are few comparable alternatives to 2D gels for tracking changes in protein expression patterns related to disease progression. The introduction of high sensitivity fluorescent staining, digital image processing and computerized image analysis has greatly amplified and simplified the detection of unique species and the quantification of proteins. By using known protein standards as landmarks within each gel run, computerized analysis can detect unique differences in protein expression and modifications between two samples from the same individual or between several individuals.

Sample Collection and Preparation

Serum samples were prepared from blood acquired by venipuncture. The blood was centrifuged at 500×g for 10 minutes, and the separated serum was divided into aliquots, and frozen at −40° C. or below until shipment. Samples were shipped on dry ice and were delivered within 24 hours of shipping.

Once the serum samples were received, logged in, and assigned a sample number, they were further processed in preparation for 2D gel electrophoresis. All samples were stored at −40° C. or below. When the serum samples were removed from storage, they were placed on ice for thawing and kept on ice for further processing.

To each 100 μl of sample, 100 μl of LB-2 buffer (5M urea, 2M Thiourea, 0.5% ASB-14, 0.25% CHAPS, 0.25% Tween-20, 5% glycerol, 100 mM DTT, 1× Protease inhibitors, and 1× Ampholyte pH 3-10) was added and the mixture vortexed. The sample was incubated at room temperature for about 5 minutes.

Separation of Proteins in Patient Samples

The proteins in the patient and control samples were separated using various techniques known in the art for separating proteins, techniques that include but are not limited to gel filtration chromatography, ion exchange chromatography, reverse phase chromatography, affinity chromatography, or any of the various centrifugation techniques well known in the art. In some cases, a combination of one or more chromatography or centrifugation steps may be combined via electrospray or nanospray with mass spectroscopy or tandem mass spectroscopy, or any protein separation technique that determines the pattern of proteins in a mixture either as a one-dimensional, two-dimensional, three-dimensional or multi-dimensional pattern or list of proteins present.

Two-Dimensional Gel Electrophoresis of Samples

Preferably the protein profiles of the present invention are obtained by subjecting biological samples to two-dimensional (2D) gel electrophoresis to separate the proteins in the biological sample into a two-dimensional array of protein spots.

Two-dimensional gel electrophoresis is a useful technique for separating complex mixtures of proteins and can be performed using a variety of methods known in the art (see, e.g., U.S. Pat. Nos. 5,534,121; 6,398,933; and 6,855,554).

Preferably, the first dimensional gel is an isoelectric focusing gel and the second dimension gel is a denaturing polyacrylamide gradient gel.

Proteins are amphoteric, containing both positive and negative charges and Like all ampholytes exhibit the property that their charge depends on pH. At low pH (acidic conditions), proteins are positively charged while at high pH (basic conditions) they are negatively charged. For every protein there is a pH at which the protein is uncharged, the protein's isoelectric point. When a charged molecule is placed in an electric field it will migrate towards the opposite charge.

In a pH gradient such as those used in the present invention, a protein will migrate to the point at which it reaches its isoelectric point and becomes uncharged. The uncharged protein will not migrate further and stops. Each protein will stop at its isoelectric point and the proteins can thus be separated according to charge. In order to achieve optimal separation of proteins, various pH gradients may be used. For example, a very broad range of pH, from about 3 to 11 or 3 to 10 can be used, or a more narrow range, such as from pH 4 to 7 or 5 to 8 or 7 to 10 or 6 to 11 can be used. The choice of pH range is determined empirically and such determinations are within the skill of the ordinary practitioner and can be accomplished without undue experimentation.

In the second dimension, proteins are separated according to molecular weight by measuring mobility through a uniform or gradient polyacrylamide gel in the detergent sodium dodecyl sulfate (SDS). In the presence of SDS and a reducing agent such as dithiothreitol (DTT), the proteins act as though they are of uniform shape with the same charge to mass ratio. When the proteins are placed in an electric field, they migrate into and through the gel from one edge to the other. As the proteins migrate though the gel, individual proteins move at different speeds with the smaller ones moving faster than the larger ones. This process is stopped when the fastest moving components reach the other side of the gel. At this point, the proteins are distributed across the gel with the higher molecular weight proteins near the origin and the low molecular weight proteins near the other side of the gel.

It is well known in the art that various concentration gradients of acrylamide may be used for such protein separations. For example, a gradient of from about 5% to 20% may be used in certain embodiments or any other gradient that achieves a satisfactory separation of proteins in the sample may be used. Other gradients would include but not be limited to from about 5 to 18%, 6 to 20%/o, 8 to 20%, 8 to 18%, 8 to 16%, 10 to 16%, or any range as determined by one of skill.

The end result of the 2D gel procedure is the separation of a complex mixture of proteins into a two dimensional array based on their unique characteristics of isoelectric point and molecular weight.

2D Gel Standards

Purified proteins having known characteristics are used as internal and external standards and as a calibrator for 2D gel electrophoresis. The standards consist of seven reduced, denatured proteins that can be run either as spiked internal standards or as external standards to test the ampholyte mixture and the reproducibility of the gels. A set mixture of proteins (the “standard mixture”) is used to determine pH gradients and molecular weights for the two dimensions of the electrophoresis operation. Table A lists the isoelectric point (pI) values and molecular weights for the proteins included in this standard mixture.

TABLE A

| Protein | pI | Molecular Weight (Da) |
|---|---|---|
| Hen egg white conalbumin | 6.0, 6.3, 6.6 | 76,000 |
| Bovine serum albumin | 5.4, 5.5, 5.6 | 66,200 |
| Bovine muscle actin | 5.0, 5.1 | 43,000 |
| Rabbit muscle GAPDH | 8.3, 8.5 | 36,000 |
| Bovine carbonic anhydrase | 5.9, 6.0 | 31,000 |
| Soybean trypsin inhibitor | 4.5 | 21,500 |
| Equine myoglobin conalbumin | 7.0 | 17,500 |

In addition, Precision Plus Protein Standards (Bio-Rad Laboratories), a mixture of 10 recombinant proteins ranging from 10-250 kD, are typically added as external molecular weight standards for the second dimension. The Precision Plus Protein Standards have an r² value of the R_{f} vs. log molecular weight plot of >0.99.

Separation of Proteins in Serum Samples

An appropriate amount of isoelectric focusing (IEF) loading buffer (LB-2), was added to the diluted serum sample, incubated at room temperature and vortexed periodically until the pellet was dissolved to visual clarity. The samples were centrifuged briefly before a protein assay was performed on the sample.

Approximately 100 μl of the solubilized protein pellet was suspended in a total volume of 184 μl of IEF loading buffer and 1 μl Bromophenol Blue. Each sample was loaded onto an 11 cm IEF strip (Bio-Rad Laboratories), pH 5-8, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min at 250V followed by a linear voltage increase to 8000V over 2.5 hours. A final rapid focusing was performed at 8000V until 20,000 volt-hours were achieved. Running the IEF strip at 500V until the strips were removed finished the isoelectric focusing process.

Isoelectric focused strips were incubated on an orbital shaker for 15 min with equilibration buffer (2.5 ml buffer/strip). The equilibration buffer contained 6M urea, 2% SDS, 0.375M HCl, and 20% glycerol, as well as freshly added DTT to a final concentration of 30 mg/ml. An additional 15 min incubation of the IEF strips in the equilibration buffer was performed as before, except freshly added iodoacetamide (C₂H₄INO) was added to a final concentration of 40 mg/ml. The IPG strips were then removed from the tray using clean forceps and washed five times in a graduated cylinder containing the Bio Rad Laboratories running buffer 1× Tris-Glycine-SDS.

The washed IEF strips were then laid on the surface of Bio Rad pre-cast CRITERION SDS-gels 8-16%. The IEF strips were fixed in place on the gels by applying a low melting agarose. A second dimensional separation was applied at 200V for about one hour. After running, the gels were carefully removed and placed in a clean tray and washed twice for 20 minutes in 100 ml of pre-staining solution containing 10% methanol and 7% acetic acid.

Staining and Analysis of the 2D Gels

Once the 2D gel patterns of the serum samples were obtained, the gels were visualized with either a fluorescent or colored stain. SyproRuby™ (Bio-Rad Laboratories) was the preferred stain. Once the protein spots had been stained, the gel was scanned and a digital image of the protein expression profile of the sample was obtained.

The digital image of the scanned gel was processed using PDQuest™ (Bio-Rad Laboratories) image analysis software to first locate the selected biomarkers and then to quantitate the protein in each of the selected spots. The scanned image was cropped and filtered to eliminate artifacts using the image editing control. Individual cropped and filtered images were then placed in a matched set for comparison to other images and controls.

This process allowed quantitative and qualitative spot comparisons across gels and the determination of protein biomarker molecular weight and isoelectric point values. Multiple gel images were normalized to allow an accurate and reproducible comparison of spot quantities across two or more gels. The gels were normalized using the “total of all valid spots method” which assumes that few protein spots change between serum samples, and that changes average out across the whole gel. The quantitative amount of the selected biomarkers present in each sample was then exported for further analysis using statistical programs. Initial Biomarker Selection

The 2D gel patterns of 33 serum samples collected from normal control subjects were compared with each other. The 33 normal control samples all gave similar 2D gel protein patterns. The normal protein expression pattern was then compared to the gel patterns obtained in serum samples of 40 patients diagnosed with AD. The comparison of the protein expression pattern of normals and AD patients identified at least 47 protein spots seen on 2D gels that differed in protein concentration.

Once the 33 normal serum samples, the 40 AD samples, and the 24 AD-Like disorder serum samples had been run in triplicates on 2D gels and an initial 47 identified protein spots were quantitated in each serum sample, the results were analyzed using statistical programs to determine which biomarkers to include in the assay for AD in order for the assay to have a sensitivity, specificity, and positive and negative predictive values to be of clinical use to physicians.

Initially, the mean and standard deviations of the biomarkers were used to select the biomarkers and to assess the statistical significance of concentration differences in the biomarkers between the control sera and the Alzheimer's disease sera. However because of the number of biomarkers studied, subsequent studies used multi-variant statistical programs to select the biomarkers. A linear discriminate functional analysis was initially employed to determine the sensitivity, specificity, positive predictive value (PPV), and negative predictive value (NPV) of each biomarker and a number of combinations of biomarkers in determining the difference between normal serum and serum taken from patients diagnosed with Alzheimer's disease. Both linear and quadratic discriminant analyses were used for statistical comparison and classification of samples.

Biostatistical Discriminant Function

The quantitative amount of the selected biomarkers present in each sample was then analyzed using a biostatistical discriminant function. The concentrations for the set of selected biomarkers were entered into a biostatistical algorithm and the sample was classified as either AD or as an AD-Like disorder or normal based on a comparison to a database of values collected from the individuals in the data set from which the discriminant function was derived.

The output of discriminant analysis is a classification table that permits the calculation of clinical sensitivity, specificity, positive predictive value (PPV) and negative predictive value (NPV). These terms are defined herein as follows: (1) the clinical sensitivity measured how often the test yielded positive results in diseased patients, in the case of the present invention, patients with AD; (2) the clinical specificity measured how often the test gave negative results in non-diseased individuals, in this case patients with AD-Like disorders or non-demented normals; (3) the negative predictive value (NPA) measured the probability that the patient would not have the disease and therefore have an AD-Like disorder or be a non-demented control when values were restricted to all individuals who tested negative; and (4) the positive predictive value (PPV) measured the probability that the patient had the disease (i.e., AD) when values were restricted to those individuals who tested positive.

A standard discriminant function analysis was performed to determine the subset of biomarkers that would be most useful in differentiating individuals with AD from those individuals with AD-Like disorders. Discriminant analysis has been well validated as a multivariate analysis procedure. Discriminant analysis identified sets of linearly independent functions that successfully classify individuals into a well-defined collection of groups. The statistical model used assumed a multivariate normal distribution for the set of biomarkers identified from each disease group.

Where xᵢⱼ represented the p-tuple vector of biomarkers from the i^{th} patient in the j^{th} group, j=1,

$$\frac{{n}_{1} + {n}_{2}}{{n}_{1} {n}_{2}} \frac{{[ {\underline{a}}^{′} ( {\overline{x}}_{1} - {\overline{x}}_{2} ) ]}^{2}}{{\underline{a}}^{′} S \underline{a}}$$

and represented the p-tuple centroid of the j^{th} group, made up of the mean biomarker values from the j^{th} disease group, then S represented the estimate of the within group variance-covariance matrix. The discriminant function was then that set of linear functions determined by the vector a. that maximizes the quantity:

$${\overline{{x}_{…}}}_{j} ,$$

The outcome of the discriminant analysis was a collection of m-1 linear functions of the biomarkers (m) that maximized the ability to separate individuals into disease groups. The vector α is the p-tuple vector which contained the coefficients that, when multiplied by an individual's biomarkers, produced the linear discriminant function, or index that was used to classify that individual. In general, if m biomarkers are used, a maximum of (m-1, g-1) discriminant functions are determined where g represented the number of groups.

Where aⱼ (k) represented the k^{th} p-tuple discriminant function. Then the value of that discriminator for the j^{th} patient is aⱼ (k)′ xᵢ. Thus for each patient there are k such values computed, which are used in a classification analysis. The discriminant functions themselves are linearly independent (i.e., for each pair of the m discriminant functions) aⱼ (k) and aⱼ (l), then aⱼ (k)′ aⱼ (l)=0. Thus, the m-1 discriminant functions provide incremental and non-redundant discriminant ability.

Identifying the discriminant function involved identifying the coefficients λ from the linear algebraic system of equations |H−λₗ (H+E)|=0 where H and E were the one way analysis of variance hypotheses and error matrices respectively. It is this computation that was provided by SAS™ statistical software. The SAS software program identified the collection of best discriminators using a forward entry procedure where the p-value to enter and the p-value to stay in the model are each 0.15.

While the discrimination procedure was fairly robust in the presence of mild departures from the normality assumption, it was very sensitive to the assumption of homogeneity of variance. This means that the variance-covariance matrices of the groups between which discrimination was sought must be equal. In this circumstance, these variance-covariance matrices can be pooled. However, in the situation where the variance-covariance matrices are not equal (multivariate heteroscedasticity), this pooling procedure is suboptimal. In this circumstance, the individual variance-covariance matrices have been used.

The use of the two within-group variance-covariance matrices is an important complication in the computation of discriminant functions. When the homoscedasticity assumption is appropriate, the within group variance-covariance matrices can be pooled, producing a linear discriminant function. The use of the within-group variance-covariance matrices produced a quadratic discriminant function (i.e., where the discriminant function is a function of the squares of the proteomic measures).

Classification Analysis

The most widely used cognitive assessment tool in primary care settings is the Mini-Mental State Examination (MMSE) score. It provides a brief evaluation of the cognitive domains affected in Alzheimer's disease, including orientation, registration, attention, recall, language and constructional praxis. MMSE has been used to detect impairment, follow the course of an illness, and monitor response to treatment. It has also been used as a research tool to screen for cognitive disorders in epidemiological studies and follow cognitive changes in clinical trials. Patients' scores range from 0 to 30, with low scores indicating greater cognitive impairment. Scores less than 24 are conventionally interpreted as evidence of a dementing illness.

Discriminant analysis was applied to the data set, from which the contribution of each individual biomarker was determined. The SAS™ statistical software program was then used to determine the linear combinations of biomarkers that provided an optimum classification of individuals into disease groups. Alternatively, the programmer can manually select different combinations of biomarkers to be incorporated into a quadratic discriminant function to optimize the classification of individuals into disease groups.

Forty seven [47] protein biomarkers were identified in the data set that both individually and/or jointly discriminated AD patient samples from samples taken from patients with AD-Like disorders. Individuals were classified as AD or having AD-Like disorders based on clinical symptoms and family history.

2D Gel Electrophoretic Controls

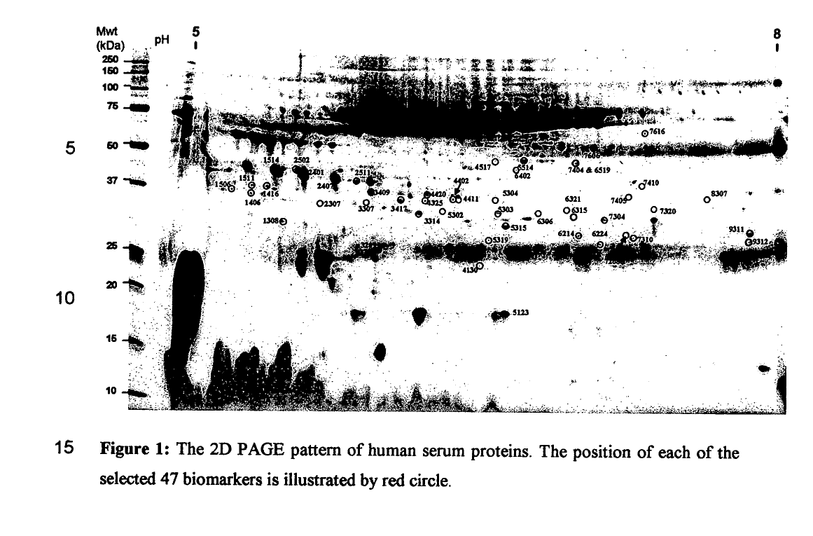

Representative samples from individuals with known cases of AD and AD-Like disorders were run as positive and negative reference controls. Serum containing all of the selected biomarkers was also provided as a reference standard. A reference control was periodically run as an external standard and for tracking overall performance and reproducibility. In addition, 2D gel images from samples classified as AD and AD-Like disorders were used for reference. The spot locations for the selected biomarkers were illustrated in FIG. 1.

The Reproducibility of Biomarker Identification and Quantification

The consistency and reproducibility of quantifying biomarkers using 2D-gel electrophoresis was characterized. To optimize reproducibility, each sample was preferably run in triplicate and each set of replicate samples was analyzed as a group. This maximized the overall accuracy of spot identification and biomarker quantification. The average percent Co-efficient of Variation (% CV) is 11±7% for 10 biomarkers quantified from a single image scanned 10 times. The average % CV is 23% for a set of biomarkers quantified from 12 separate processed aliquots of the same sample. The range in biomarker concentrations for this group of biomarkers ranged from a low of 248 ppm to a high of 15,548 ppm normalized concentration of spot per total detected spots in the 2D gel.

The protein concentrations employed in the discriminant function were relative values obtained by normalizing the intensity of each spot to all detected spots in 10 the image. The linear range in protein concentrations was 0.5 to 1,000 ng per spot. The concentration of any given spot was the absolute amount of protein in that spot divided by the total protein loaded onto the gel. The total amount of protein loaded onto a gel was typically about 100 μg.

Serum is primarily comprised of a highly conserved distribution of the most abundant proteins, such as albumin and immunoglobulin, which enhance efforts to ensure the reproducibility and consistency of biomarker detection and quantitation. The selected biomarkers represented a minor fraction of the total serum protein. Therefore the concentration of the selected biomarkers varied significantly as a function of disease state without significantly shifting the overall distribution and concentration of the major serum proteins. Discriminant biostatistics were employed to establish the dynamic concentration range of the selected biomarkers useful in differentiating AD patients.

Biomarker Stability

The effect of multiple freeze/thaw cycles on protein stability and sample 25 integrity was investigated. A serum sample was collected and aliquoted. One aliquot was processed without freezing, while other aliquots were frozen at −80° C. and thawed repetitively. A second set of serum samples was diluted into loading buffer and aliquoted. The second set of samples, similar to the first set, had one aliquot processed without freezing and other aliquots frozen at −80° C. and thawed repetitively.

Triplicate samples were processed as described. The scanned images of the 2D gels were analyzed, and the quantities of each of the 47 neurodegenerative biomarkers of interest were determined. The results illustrated that freezing and thawing either undiluted or diluted serum samples up to 10 times had no significant effect on the serum protein profile or on the abundance of the selected biomarkers.

In addition, sample deterioration was investigated over a one-year period. Twenty-one selected biomarkers were quantitated in control samples stored at −80° C. An aliquot of each control sample was processed several times each quarter, or each 3 month time period. The results demonstrated that there was no significant increase or decrease in the quantity of biomarker detected over a one-year time frame for samples stored at −80° C., beyond that which is typically observed for processing replicate samples.

Samples Analyzed

Serum samples were obtained from 40 AD patients and 24 patients having AD-Like disorders. All individuals with symptoms of a neurodegenerative disorder were evaluated and diagnosed by a neurologist. Clinical evaluation was performed to classify individuals into one of seven stages of AD. Stages 1 to 7 are defined as follows:

(a) Stage 1 individuals showed no cognitive impairment;

(b) Stage 2 individuals had a very mild cognitive decline with memory lapses that were not necessarily evident during a medical examination or apparent to friends, family, or co-workers;

(c) Stage 3 individuals had a mild cognitive decline with some patient's, but not all, memory or concentration problems measurable in clinical testing or detailed medical interviews;

(d) Stage 4 individuals had a moderate cognitive decline with clear-cut deficiencies in knowledge of recent occasions or current events and an impaired ability to perform challenging mental arithmetic detectable in careful medical interviews;

(e) Stage 5 individuals had a moderately severe cognitive decline with the patient exhibiting major gaps in memory and deficits in cognitive function and where some assistance with day-to-day activities becomes essential;

(f) Stage 6 individuals had a severe cognitive decline with significant memory difficulties and potential changes in personality, with Stage 6 individuals tending to wander and become lost and needing extensive help with daily activities; and

(g) Stage 7 individuals had a very severe cognitive decline where muscle reflexes become abnormal and the muscles grow rigid as exhibited in the loss of the ability to walk without assistance, to sit without support, to smile, to hold the head up, and/or to swallow normally.

The AD-Like disorders included individuals with the following conditions: Parkinson's disease, Lewy body dementia, Multi-infarct dementia, Alcohol-related dementia, Post-radiation Encephalopathy, Seizures, Memory dysfunction, Semantic dementia, Chronic inflammatory demyelinating polyneuropathy (CIDP), Cerebrovascular accident (CVA) dementia, Thalamic CVA, Frontotemporal dementia (FTD) and Corticalbasal danglionic degeneration (CBGD).

Differentiating AD, AD-Like Disorders, and Normals

The preferred embodiment used all 47 biomarkers of interest. Although a variety of different combinations of biomarkers were also tested that gave comparable statistical performance, they are not specifically described herein but would be performed in a similar fashion.

As shown in FIG. 1, the serum proteins were resolved by 2D gel electrophoresis of human serum proteins. The proteins were visualized by the sensitive (≦1 ng protein/spot out of 100 μg serum proteins per gel) and linearly staining (linearity and dynamic range of from ≦1 ng to ≧100 ng) SyproRuby™ fluorescent stain. The stained gels were scanned and the digital image of the 2D gel was analyzed using PDQuest™ quantitative digital image analysis software.

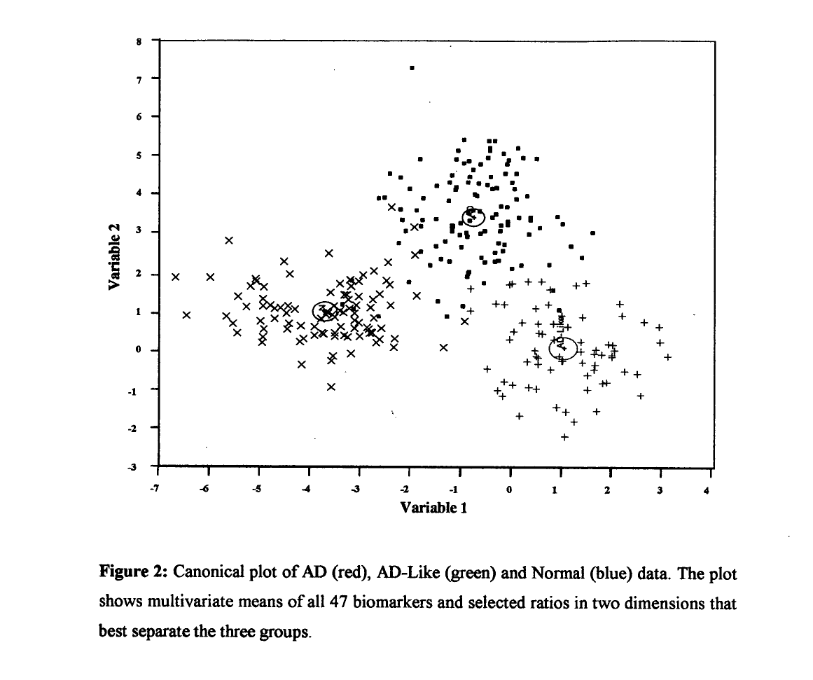

Quantitative level of all 47 biomarkers (Table 1) and selected ratios (Table 2) were tabulated as mean±SD for AD, AD-Like and Normal subjects. Data of all biomarkers of AD, AD-Like and Normal was then used to create a canonical plot (FIG. 2). The data represent the multivariate means of all 47 biomarkers and selected ratios plot in two dimensions that best separate the three groups. The quantitative results were then subjected to linear and quadratic discriminant analysis using the SAS™ statistical software. The results are shown in Tables 2 and 3. The linear discriminant analysis correctly discriminate AD patient with sensitivity of 91.6% from AD-Like patients (90.3% specificity) while quadratic analysis classified AD patients from AD-Like patient with sensitivity and specificity of 100%. When Normal samples are included the linear discriminant analysis correctly distinguished AD and AD-Like patients with sensitivity of 88.2% and 81.9%, respectively, from normal individuals (92.9% specificity). Quadratic analyses distinguish the AD and AD-Like patients at sensitivity of 98.3% and 100%, respectively, from Normal (100% specificity).

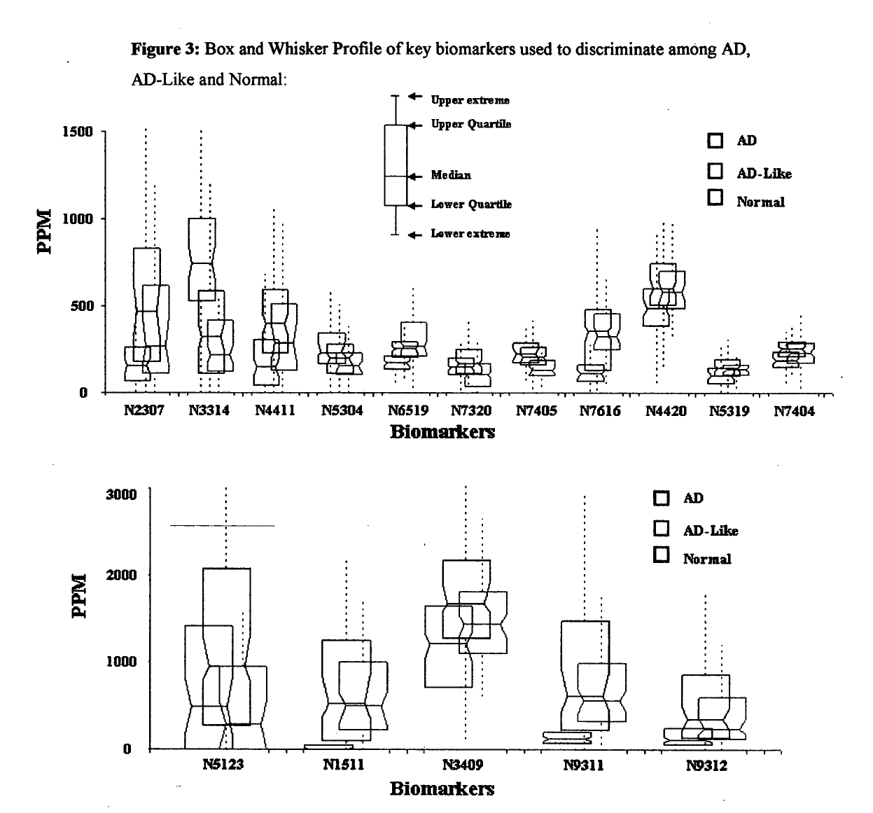

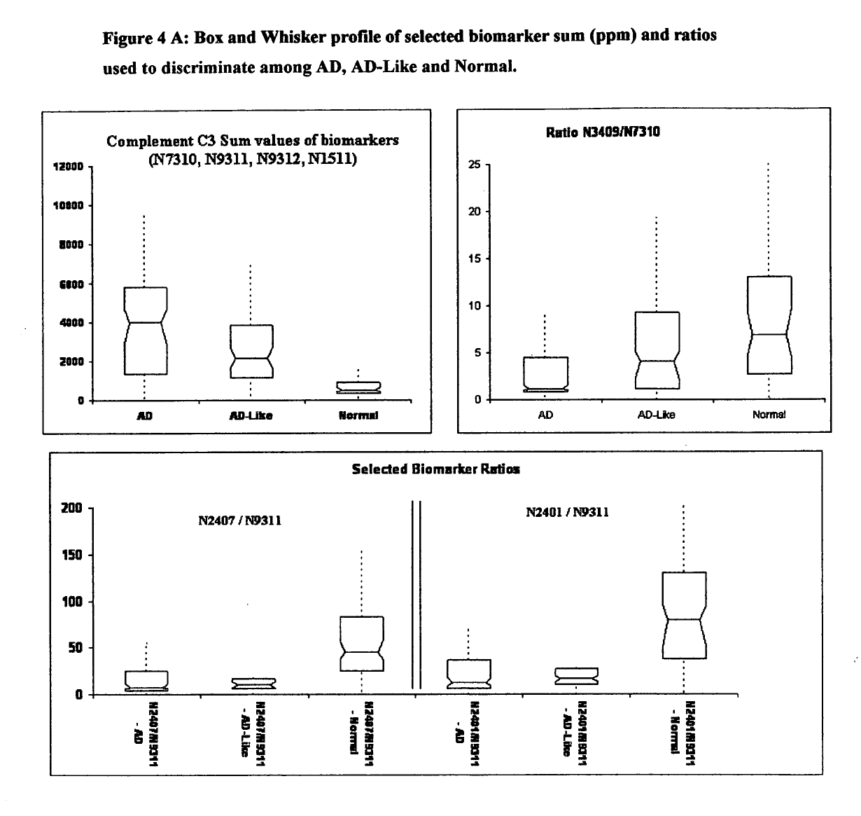

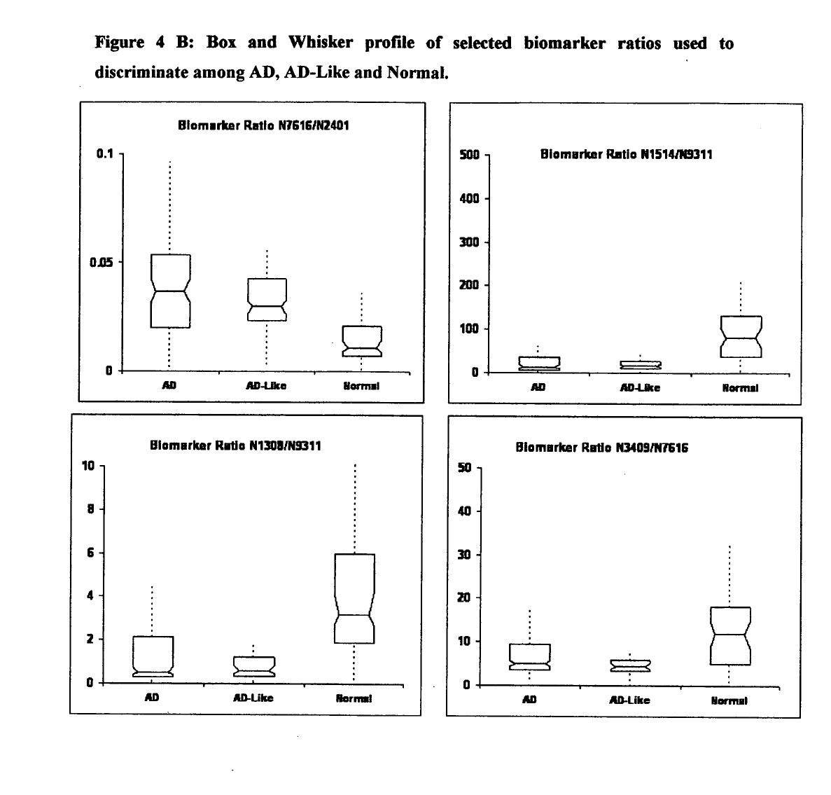

The Box and Whisker plot profile of key biomarkers and selected biomarker ratios used in the discriminant analysis are presented in FIGS. 3, 4A and 4B. These plots show the median (the central line in the box) of each selected markers or ratios. A Box and Whisker plot is a way of summarizing a set of data measured on an interval scale. It is often used in explanatory data analysis. This type of graph is used to show the shape of the distribution of the data, its central value, and its variability. The upper line of the box represents the upper quartile range of data while the bottom line of the box represents the lower quartile range of the data. The whiskers are the two lines outside the box that extend to the highest and lowest observations.

Assay for AD vs. AD-Like Disorders

Definitive diagnostic tests to confirm the diagnosis of Alzheimer's disease (AD) and distinguish it from the AD-Like disorders that display similar symptoms but have different treatment options and prognoses have long been sought by clinicians in hopes of providing earlier treatment decisions and improved patient outcomes. AD is a progressive brain disorder that gradually destroys a person's memory and ability to learn, reason, make judgments, communicate and carry out daily activities. As AD progresses, individuals may also experience changes in personality and behavior, such as anxiety, suspiciousness or agitation, as well as delusions or hallucinations.

Presently, the diagnosis of AD is a clinical one. There is no test that provides diagnostic certainty. The usual diagnostic process consists of a full medical history, and comprehensive physical and neurological examinations. In addition to neurological examinations, patients are generally tested with a standardized cognitive screening test, such as the Mini Mental State Examination (MMSE.)

In summary, the diagnosis of AD is currently based on clinical criteria and the results of cognitive screening tests. Neuroimaging and blood studies are often performed, mostly to rule out the presence of other medical conditions that may mimic the clinical appearance of AD. To date no definite biochemical or genetic test is available to definitively diagnose AD, or to differentiate AD from AD-Like disorders and/or from normals.

The present invention provides an objective biochemical assay for differentiating AD from AD-Like disorders and from normals. The assay is comprised of the following steps: (1) collecting a serum sample from a patient; (2) running triplicate 2D gel electrophoreses of the patient sample; (3) staining the 2D gel; (4) creating a digital image of the 2D gel; (5) quantifying the protein concentration in selected protein spots on the 2D gel; and (6) performing a statistical analysis on the quantity of the selected proteins to determine the Likelihood of the patient having AD or an AD-Like disorder.

While the methods have been described in terms of preferred embodiments, it will be apparent to those of skill in the art that variations may be applied to the methods including the sequence of steps in the methods. Certain agents may be substituted by one of skill and similar results may be achieved, as will be appreciated by one of skill in the art. Such modifications or substitutions to the methods of the present invention are deemed to be within the spirit, scope and concept of the invention as defined by the disclosure and its claims.

TABLE 1
Mean ± Standard Deviation (SD) of the 47 biomarkers in the sera of AD,
AD-Like and Normal individuals. Values are expressed as PPM

| | Diagnosis | | |
|---|---|---|---|
| | AD | AD-LIKE | NORMAL |
| Biomarker | MEAN ± SD | MEAN ± SD | MEAN ± SD |
| N2307 | 533.1 ± 398.1 | 368.1 ± 317.8 | 235.0 ± 325.3 |
| N3307 | 265.3 ± 579.2 | 1255.0 ± 2775.0 | 299.9 ± 131.3 |
| N3314 | 421.8 ± 418.6 | 293.1 ± 280.2 | 803.9 ± 481.7 |
| N4411 | 446.9 ± 347.4 | 347.3 ± 307.8 | 234.0 ± 357.0 |
| N5123 | 1473.0 ± 1810.7 | 1089.6 ± 2102.7 | 975.9 ± 1445.5 |
| N5302 | 119.0 ± 201.2 | 28.8 ± 101.9 | 127.6 ± 237.9 |
| N5303 | 468.5 ± 174.7 | 421.7 ± 187.8 | 479.5 ± 188.9 |
| N5304 | 204.7 ± 124.6 | 162.4 ± 88.8 | 270.3 ± 162.3 |
| N6214 | 436.6 ± 198.0 | 514.7 ± 776.4 | 455.9 ± 150.5 |
| N6224 | 381.7 ± 272.6 | 332.6 ± 193.6 | 299.9 ± 148.8 |
| N6306 | 204.9 ± 78.2 | 212.6 ± 140.4 | 206.3 ± 70.6 |
| N6321 | 92.2 ± 56.3 | 187.2 ± 363.0 | 86.8 ± 42.8 |
| N6402 | 408.5 ± 244.7 | 319.6 ± 234.8 | 372.7 ± 185.6 |
| N6519 | 242.1 ± 86.7 | 338.6 ± 236.4 | 181.1 ± 56.3 |
| N7208 | 84.4 ± 160.8 | 56.8 ± 75.7 | 59.6 ± 73.4 |
| N7304 | 755.8 ± 277.2 | 650.1 ± 337.0 | 1062.2 ± 868.4 |
| N7310 | 1461.7 ± 1221.8 | 852.9 ± 1035.3 | 358.2 ± 850.7 |
| N7320 | 180.0 ± 81.7 | 122.2 ± 112.9 | 159.7 ± 90.0 |
| N7405 | 219.5 ± 93.4 | 143.9 ± 61.5 | 228.9 ± 75.1 |
| N7410 | 99.2 ± 115.0 | 41.5 ± 29.8 | 38.4 ± 20.1 |
| N7616 | 346.0 ± 246.1 | 339.6 ± 146.7 | 161.6 ± 348.1 |
| N8307 | 80.4 ± 61.8 | 74.2 ± 42.6 | 69.6 ± 40.6 |
| N1308 | 486.4 ± 267.5 | 368.8 ± 194.7 | 445.5 ± 153.2 |
| N1406 | 554.8 ± 223.2 | 448.0 ± 164.5 | 481.5 ± 164.7 |
| N1416 | 473.2 ± 210.7 | 436.0 ± 290.5 | 379.8 ± 192.9 |
| N1506 | 584.4 ± 404.4 | 466.7 ± 299.2 | 551.6 ± 201.9 |
| N1511 | 750.0 ± 687.9 | 599.5 ± 448.1 | 92.8 ± 360.5 |
| N1514 | 9204.9 ± 3421.4 | 10838.3 ± 3978.7 | 9774.2 ± 3566.1 |
| N2401 | 9651.8 ± 3341.5 | 11310.3 ± 4939.9 | 9393.0 ± 3228.3 |
| N2407 | 6173.7 ± 2321.0 | 7172.2 ± 4043.1 | 6158.2 ± 2675.0 |
| N2502 | 1874.4 ± 1191.1 | 1836.0 ± 1112.3 | 2273.1 ± 943.0 |
| N2511 | 689.4 ± 308.8 | 672.5 ± 488.1 | 688.4 ± 291.6 |
| N3209 | 1647.1 ± 588.1 | 1807.7 ± 812.7 | 1647.2 ± 527.3 |
| N3409 | 1761.4 ± 774.9 | 1545.6 ± 759.1 | 1304.0 ± 747.9 |
| N3417 | 430.7 ± 246.2 | 388.4 ± 155.7 | 343.0 ± 102.4 |
| N4130 | 259.2 ± 108.0 | 266.4 ± 191.9 | 320.5 ± 197.3 |
| N4325 | 692.0 ± 176.8 | 572.9 ± 162.9 | 627.8 ± 184.9 |
| N4402 | 364.7 ± 854.0 | 191.0 ± 352.6 | 201.2 ± 224.4 |
| N4420 | 642.5 ± 263.3 | 614.7 ± 247.6 | 478.8 ± 187.7 |
| N4517 | 85.4 ± 109.9 | 167.8 ± 318.1 | 167.3 ± 324.3 |
| N5315 | 858.6 ± 232.4 | 812.8 ± 217.9 | 861.0 ± 300.1 |
| N5319 | 147.0 ± 74.1 | 136.7 ± 53.3 | 150.2 ± 177.2 |
| N5514 | 559.1 ± 228.2 | 526.8 ± 269.0 | 558.0 ± 267.6 |
| N6315 | 82.2 ± 41.8 | 87.4 ± 50.8 | 93.4 ± 45.3 |
| N7404 | 246.9 ± 79.6 | 246.6 ± 117.1 | 435.8 ± 812.5 |
| N7606 | 2778.1 ± 1133.2 | 2709.7 ± 1099.3 | 2693.4 ± 1174.8 |
| N9311 | 923.9 ± 835.1 | 665.1 ± 481.7 | 205.0 ± 364.2 |
| N9312 | 592.6 ± 633.8 | 376.6 ± 353.9 | 315.5 ± 498.0 |

TABLE 2
Mean ± Standard Deviation (SD) of selected biomarker sum and ratios
used in the discriminant function analysis.

| | Diagnosis | | |
|---|---|---|---|
| | AD | AD-LIKE | NORMAL |
| Biomarkers | MEAN ± SD | MEAN ± SD | MEAN ± SD |
| N1308/N9311 | 17.2 ± 102.0 | 1.4 ± 2.3 | 5.1 ± 8.5 |
| N2407/N9311 | 147.4 ± 838.3 | 28.8 ± 55.9 | 73.6 ± 130.0 |
| N2401/N9311 | 205.2 ± 1144.6 | 42.7 ± 74.7 | 111.5 ± 196.2 |
| N1514/N9311 | 162.8 ± 871.8 | 39.9 ± 62.3 | 115.0 ± 201.3 |
| N3409/N7310 | 85.1 ± 382.9 | 80.2 ± 305.4 | 226.8 ± 620.2 |
| N7616/N2401 | 1.2 ± 8.1 | 4.1 ± 20.2 | 0.0 ± 0.0 |
| N3409/N7616 | 12.0 ± 17.6 | 5.3 ± 4.9 | 14.4 ± 13.4 |
| C3 Sum | 3728.4 ± 2493.0 | 2494.3 ± 1630.3 | 972.3 ± 1659.1 |

TABLE 3
Discriminant Analysis of AD (n = 40) and AD-Like (n = 24)
patients using 47 biomarkers:

| | |
|---|---|
| Linear Discriminant | Quadratic Discriminant |
| Analysis | Analysis |

| Sensitivity | Specificity | Sensitivity | Specificity |
|---|---|---|---|
| 91.6% | 90.3% | 100.0% | 100.0% |

TABLE 4
Discriminant Analysis of AD (n = 40), AD-Like (n = 24) patients
compared to Normal (n = 33) individuals, using 47 biomarkers:

| Linear Discriminant Analysis | | | Quadratic Discriminant Analysis | | |
|---|---|---|---|---|---|
| Sensitivity | | Specificity | Sensitivity | | Specificity |
| AD | AD-Like | Normal | AD | AD-Like | Normal |
| 88.2% | 81.9% | 92.9% | 98.3% | 100.0% | 100.0% |

What is claimed is:

1. An assay for selecting appropriate biomarkers useful in diagnosis of Alzheimer's disease (AD) comprising:
a) collecting serum samples from patients with AD;
b) collecting serum samples from patients with an AD-Like disorder;
c) performing a two-dimensional gel electrophoretic analysis of the serum sample;
d) staining the two-dimensional gel;
e) quantitating a protein concentration in a plurality of protein spots on the two-dimensional gel; and
f) performing a statistical analysis on the quantities of the proteins in the protein spots to select a biomarker spot to distinguish between patient with AD from patients with the AD-Like disorder and normal controls.

2. A screening assay for Alzheimer's disease (AD) comprising:
a) collecting a serum sample from a patient;
b) performing a two-dimensional (2D) gel electrophoretic analysis of the serum sample;
c) staining the 2D gel pattern;
d) quantitating a set of preselected protein spots; and
e) performing a statistical analysis on the quantity of the selected spots to determine the Likelihood of the patient having AD.

3. An assay for differential diagnosis of Alzheimer's disease (AD) comprising:
a) collecting a serum sample from a patient;
b) performing a two-dimensional (2D) gel electrophoretic analysis of the serum sample;
c) staining the 2D gel pattern;
d) quantitating a set of preselected protein spots; and
e) performing a statistical analysis on the quantity of the selected spots to determine the Likelihood of the patient having AD as opposed to an AD-Like condition.
